(19) United States
(12) Patent Application Publication
(10) Pub. No.: US 2007/0142891 A1
(43) Pub. Date: Jun. 21, 2007

(54) Therapeutic device that provides stimulation to an immobilized extremity

(76) Inventor: Solomon Tony Stanley, Visalia, CA (US)

Correspondence Address: Robert Z. Evora, 4741 Planters Walk, Douglasville, GA, 30135, US

(21) Appl. No.: 11/346,073

(22) Filed: Feb. 2, 2006

Related U.S. Application Data
(60) Provisional application No. 60/751,357, filed on Dec. 16, 2005.

(51) Int. Cl. A61N 1/18

(52) U.S. Cl. 607/144; 607/145

(57) ABSTRACT

A therapeutic device for biasing a body portion of a patient into a predetermined position. The therapeutic device includes a rigid support that is substantially flat and contoured to brace the body portion into the predetermined position. A securing mechanism is provided to brace the rigid support against the body portion and a therapeutic stimulation mechanism is provided. The therapeutic stimulation mechanism can include an electrical stimulation mechanism or a heating mechanism to provide heating and electrical stimulation to the body portion.

### CROSS REFERENCE TO RELATED APPLICATION

This application is a Non-Provisional Application which claims the benefit of the filing date of U.S. Provisional Application Ser. No. 60/751,357, entitled “A THERAPEUTIC DEVICE THAT PROVIDES STIMULATION TO AN IMMOBILIZED EXTREMITY” filed Dec. 16, 2005, the entirety of which is incorporated herein by reference.

### BACKGROUND OF THE INVENTION

1. Field of the Invention

The present invention relates in general to a rigid therapeutic device for immobilized portions of a person's body who suffer from a lack of muscular control, and in particular to providing therapeutic stimulation with heat and/or electrical pulses.

2. Description of the Related Art

Loss of muscular control is oftentimes afflicted to individuals who have suffered from a stroke, comatose patients, palsy patients, arthritis patients, and those who have had tendon surgery. In many cases, loss of muscular control causes the patient's hands to clench shut and their feet to curl downward into a clenched configuration. When forced open, there is a tendency for their hands and feet to bias back into the unsightly stressed position. In order to reposition the hand or foot into a preferred unclenched position, the extremity must be urged back into the desired configuration. In other instances, stimulation may be provided to the damaged extremity in addition to therapy to reorient the extremity into a preferred operable position.

Attempts have been made to construct splints which allow some residual mobility. However, conventional devices fail to secure a patient's extremity in a preferred position as well as provide therapy to the damaged extremity simultaneously.

### SUMMARY OF THE INVENTION

An object of the present invention is to provide a therapeutic device for immobilized portions of a patient's body who suffers from a lack of muscular control.

Another object of the present invention is to provide therapeutic stimulation to the patient's immobilized extremity by providing heat and/or electric impulses as the stimulus.

According to this invention, a therapeutic device is provided for biasing a body portion of a patient into a predetermined position. A substantially flat rigid support that is contoured to the patient's hand is provided to brace the body portion into the predetermined position. A securing mechanism braces the rigid support against the body portion and a therapeutic stimulation mechanism is included. The therapeutic stimulation mechanism includes an electrical stimulation mechanism and a heating mechanism to provide heating and electrical stimulation to the body portion at desired amounts and and/or desired intervals.

The therapeutic stimulation mechanism is programmable and adapted to provide predetermined amounts of heating and electrical simulation to the body portion.

A further object of the present invention is to integrate the therapeutic stimulus in a glove to a patient's immobilized hand or in a booty (or sock-like covering) to the patient's immobilized foot.

Still another object of the present invention is to provide therapeutic device having a rigid contoured padded portion secured to the patient's hand or foot that serves as a splint to prevent the hand or foot from biasing into a clenched closed configuration. In addition, the therapeutic device also includes therapeutic stimulation to the patient's immobilized extremity by providing heat and/or electric impulses as the stimulus.

These and other objects, features, and/or advantages may accrue from various aspects of embodiments of the present invention, as described in more detail below.

### BRIEF DESCRIPTION OF THE DRAWINGS

Various exemplary embodiments of this invention will be described in detail, wherein like reference numerals refer to identical or similar components or steps, with reference to the following figures, wherein:

FIG. 1 is an illustration of a contoured pad on a therapeutic device for a hand according to this invention.

FIG. 2 is a side view illustration of the patient's hand fastened to the contoured pad on a therapeutic device according to this invention.

FIG. 3 is an illustration of a contoured pad on a therapeutic device for a foot according to this invention.

FIG. 4 is a side view illustration of the patient's foot fastened to the contoured pad on a therapeutic device according to this invention.

FIG. 5 is an illustration of a therapeutic device configured as a pair of gloves having a stimulation mechanism including a therapeutic heating control mechanism according to this invention.

FIG. 6 is a side view illustration of the therapeutic device secured to a contoured pad including the therapeutic heating control mechanism according to this invention.

FIG. 7 is an illustration of a therapeutic device having another stimulation mechanism including an electrical mechanism that provides a therapeutic electro-massage to stimulate the patient's hand according to this invention.

FIG. 8 is an illustration of a pair of therapeutic devices having a stimulation mechanism comprised of a pair of electrical mechanisms that provide a therapeutic electro-massage to stimulate the patient's hand according to this invention.

FIG. 9 is an illustration of the stimulation mechanism disposed in a contoured pad according to this invention.

FIG. 10 is an illustration of a stimulation mechanism disposed in the cushion disposed on a contoured pad according to this invention.

FIG. 11 is an illustration of a therapeutic device configured as a sock-like booty having a stimulation mechanism comprised of a heating control mechanism according to this invention.

FIG. 12 is an illustration of a therapeutic device configured as a sock-like booty attached to the contoured pad and including the stimulation mechanism according to this invention.

FIG. 13 is a front side illustration of a therapeutic device configured as a glove having a therapeutic stimulation mechanism with a heating control mechanism and an electric mechanism according to this invention.

FIG. 14 is a bottom side illustration of a therapeutic device configured as a glove having a therapeutic stimulation mechanism with a heating control mechanism and an electric mechanism according to this invention.

FIG. 15 is a side view illustration of a therapeutic device configured as a glove having a therapeutic stimulation mechanism with a heating control mechanism and an electric mechanism according to this invention.

### DETAILED DESCRIPTION OF EXEMPLARY EMBODIMENTS

Particular embodiments of the present invention will now be described in greater detail with reference to the figures.

This invention overcomes the conventional problems described above by providing a therapeutic device for immobilized portions of a person's extremity who suffers from a deterioration of muscular control.

Adapted for a Patient's Hand

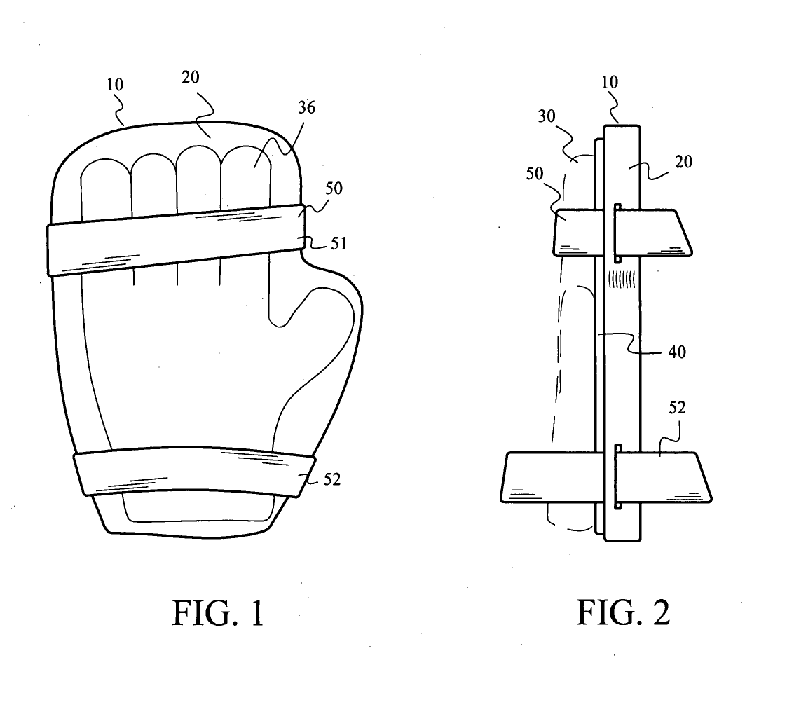

FIGS. 1 and 2 are an exemplary illustration of a therapeutic device 10 for a patient's hand 30. The therapeutic device 10 includes a contoured pad 20. The contoured pad 20 may be shaped along the outer silhouette of the hand 30 or any shape capable of restraining a portion of the patient's hand. The contoured pad 20 is made out of a rigid material to brace the hand 30. The contoured pad 20 may include any number of solid materials including a polymer, a metal, and/or any other material suitable for providing a rigid structure. The surface of the contoured pad 20 that the hand 30 lies against may be supported by a flat soft cushion 40 composed of a material such as, felt, cotton, sponge, rubber and or any other soft substance now known or later developed. The cushion 40 may be disposed on the contoured pad 20 with an adhesive, by being sewn thereon, and/or any other method for fastening the cushion 40 to the contoured pad 20. The cushion 40 and/or the contoured pad 20 may include a depression 36 that the patient's hand (as shown in FIG. 1) can be secured into to provide added comfort and to accurately position the fingers in a desired orientation.

Straps 50 are disposed on a surface of the contoured pad 20 to brace the hand 30 to the contoured pad 20. As shown in FIGS. 1-2, two straps 51, 52 are employed on the surface of the contoured pad 20. The first strap 51 fastens the patient's fingers to the contoured pad 20. A second strap 52 is shown securing the patient's wrist to the contoured pad 20. The straps 50 may include any number of mechanisms for securing, such as a buckle, Velcro, clamps, a tie or the like. Any number of straps may be applied to the upper surface of the contoured pad 20 to hold the patients hand 30 in a desired orientation. For example, numerous straps can be provided to secure each of the fingers individually in place. Alternatively, a single strap can be interwoven within the contoured pad 20, or the cushion 40 to create various loops to receive each of the fingers individually.

The contoured pad 20 may be used to bias the selected body portion, such as a hand, a finger, or a foot, to a desired secured position to aid in the healing and rehabilitation process, such as after an injury, surgery, or to assist a patient with at least partially deteriorated muscle control. This desired position may be a position which promotes proper mending of bones, healing of tendons or muscle tissue. Alternatively, it may be the desired at-rest position of the patient's body portion to overcome displacement caused by palsy, bone deformity, or other similar disablement such as a spasmed closed hand or foot.

Adapted for a Patient's Foot

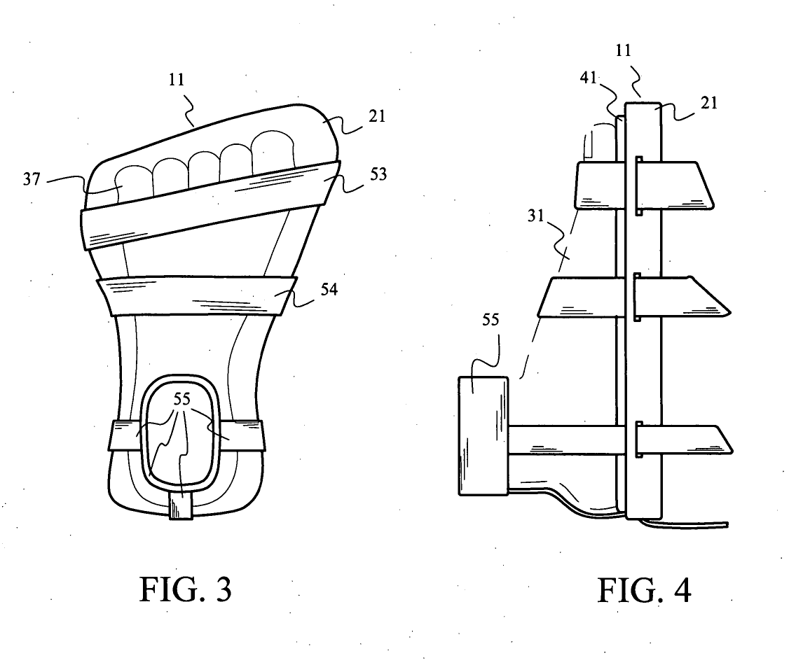

In the alternative, FIGS. 3 and 4 illustrate an exemplary design for a therapeutic device 11 adapted for a patient's foot 31. The therapeutic device 11 includes a contoured pad 21. The contoured pad 21 may be shaped along the outer silhouette of a foot 31 and/or any shape capable of restraining a portion of the patient's foot. The contoured pad 21 is also made of a solid material to brace the foot 31. The contoured pad 21 may include any number of solid materials suitable for providing a rigid structure. The surface of the contoured pad 21 that the foot 31 is braced against may also be supported by a flat soft cushion 41 as described above. The cushion 41 may be disposed on the contoured pad 21 in a variety of ways as described above with respect to the hand 30. The cushion 41 and/or the contoured pad 21 may also include a depression 37 that the patient's foot 31 can be secured into to provide added comfort and to accurately position the toes of the patient's foot 31 in a desired orientation.

Straps 53, 54, 55 are disposed on a surface of the contoured pad 21 adjacent to the foot 31. A first strap 53 fastens the patient's toes onto the contoured pad 21. A second strap 54 is shown securing the patient's forefoot to the contoured pad 21. In addition, straps 55 are provided to secure the ankle and heel of the foot 31 to the contoured pad 21. As mentioned above, the straps 53, 54, 55 may include and/or be substituted by any number of mechanisms for securing, and any number of straps may be applied to the upper surface of the contoured pad 20 to hold the patients foot 31 in a desired orientation. Numerous straps can also be provided to secure each of the toes individually in place. Alternatively, a single strap can be interwoven within the contoured pad 21 or the cushion 41 to create many loops to receive each of the patient's toes individually.

Therapeutic Heat Stimulation

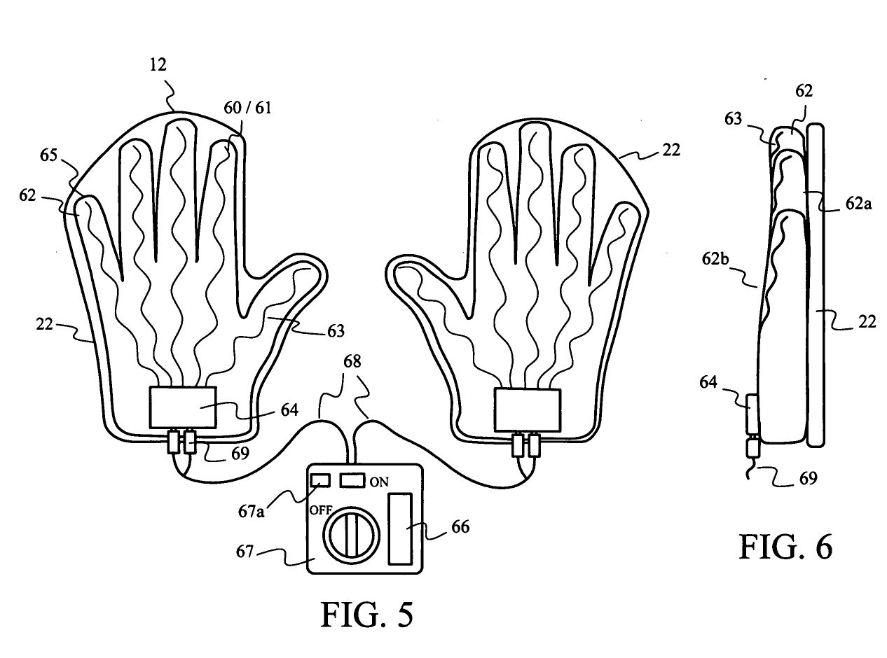

FIGS. 5-6 illustrate a therapeutic device 12 according to this invention that includes a therapeutic stimulation mechanism 60. The stimulation mechanism 60 includes a heating mechanism 61. The heating mechanism 61 includes a glove 62 that is worn on the hand 30 of a patient. The glove 62 is attached to a rigid contoured pad 22. Although the contoured pad 22 is shown disposed adjacent to the glove 62, the contoured pad 22 may be integrated within the glove 62. The rigid contoured pad 22 may be attached to either the top or bottom side of the glove 62.

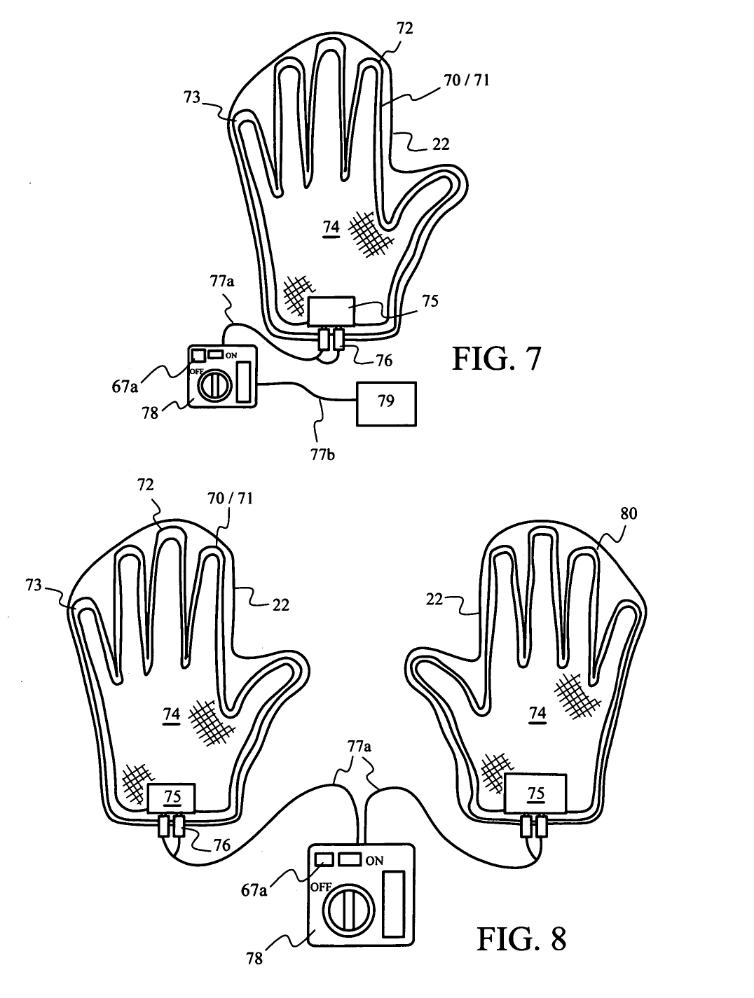

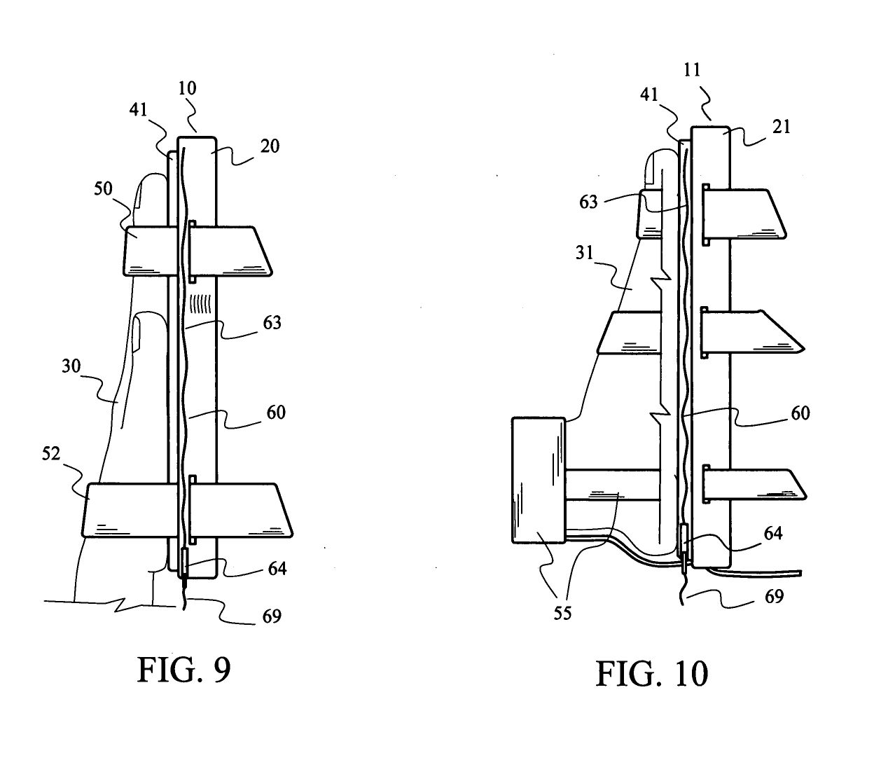

Circuitry of the heating mechanism 61 can be integrated into the contoured pad 22 (see FIG. 9 illustrating the heating therapeutic mechanism 60 disposed with the contoured pad 20) or alternatively the heating mechanism 61 may be embedded in the glove 62 (as shown in FIG. 6) without departing from the scope of the invention. The heating mechanism 61 may also be disposed within the cushion 41 (as shown in FIG. 10) without departing from the scope of the invention. In the alternative, the heating mechanism 61 may be integrated into the straps 50 (not shown) and/or any other fasteners provided to secure the hand 30 or foot 31 of the patient to the contoured pad. Similar construction applies for the electrical stimulation mechanism described in more detail below with respect to FIGS. 7 and 8.

Referring to FIG. 6, the glove 62 includes a bottom portion 62a that covers the palm side of the hand 30 and top portion 62b that covers the back side of the hand 30. The glove may be manufactured from a variety of materials, including a stretchable fabric, such as LYCRA, cotton, a polymer and or any other suitable material which closely conform to the shape of the patient's hand 30 that is adapted to support the heating mechanism 61 according to this invention.

As shown in FIG. 5, the heating mechanism 61 includes various thin wires 63 that extend into each of the fingers 65 in the glove 62 from a plug portion 64 disposed in the glove 62 adjacent to the wrist portion of the glove 62. The plug portion 64 may be embedded anywhere in the fabric of the glove 62 and is not limited to the wrist portion.

The heating mechanism 61 includes a power supply 66 attached to a heating control portion 67. The heating control portion 67 is electrically connected to the plug portion 64. The heating control portion 67 can be remotely controlled from the glove 67. Alternatively, the heating control portion 67 may be disposed on the glove and controlled at the glove 62 such that heating mechanism 61 could be a completely self-sustained heating unit. The heating mechanism 61 can be composed of a variety of methods now known or later described which operably warms the patient's hand 30 within the glove 62. For example, the glove may be comprised of a metal coated fabric inside or outside of the glove and may be connected from a wire 68 to a heating control portion 67 through the plug portion 64.

The power supply 66 may be disposed in a battery compartment of the heating control portion 67. The heating control portion 67 may be configured to have terminals connected through wires 68 to a socket 69 that mates with the plug portion 64.

In operation, electrical power is provided from power supply 66 through socket 69 to plug portion 64 to wires 63 in the glove 62 when the stimulation mechanism 60 is powered on. The amount of heat generated in the glove 62 can be controlled in a variety of known methods, for example, by modulation of the width of the drive pulses as a function of temperature. A processor 67a (as described in more detail later) may be incorporated for programmable control.

The power supply 66 may be contemplated from any number of different power sources, for example, the power source could be solar powered or some other type of battery. The power source can also be mounted in a number of other locations in accordance with this invention.

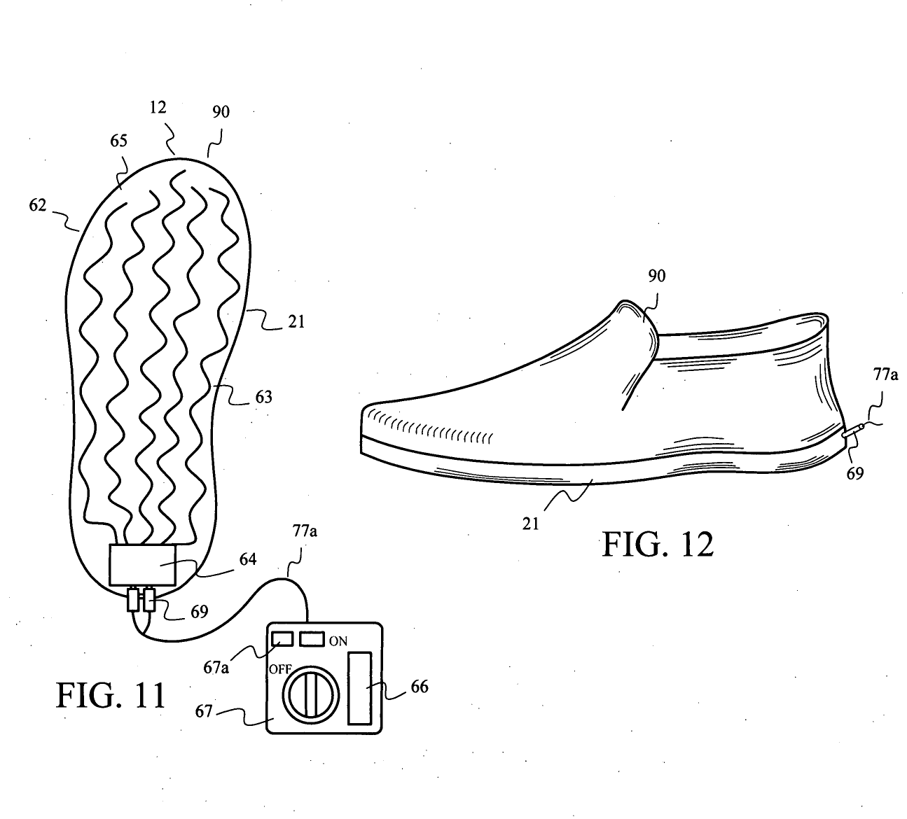

In the alternative, the stimulation mechanism 60 including the heating mechanism 61 can be incorporated into a sock-like booty 90 for the patient's foot 31 as shown in FIGS. 11 and 12 attached to the contoured pad 21. The heating mechanism 61 would operate similarly to the mechanism integrated for the patient's foot 31 as shown in FIG. 5.

Therapeutic Electrical Stimulation

FIGS. 7 and 8 illustrate another exemplary embodiment of the invention. The therapeutic stimulation mechanism 70 includes an electrical mechanism 71 that provides a therapeutic electro-massage to stimulate the patient's hand 30 or foot 31. The electrical mechanism 71 can be adapted to function in a glove 72 that is worn on the hand 30 of a patient as shown in FIG. 7. In the alternative, the circuitry of the electrical mechanism 71 can be integrated into a rigid contoured pad 22 secured to the glove 72 to operate in conjunction with the glove 72 or straps 50 where the straps serve as electrodes whose functionality will be described below.

FIG. 7 illustrates an electrode glove 72 according to this invention. The electrode glove 72 includes an outer insulating portion 73 formed of a flexible elastomeric material. The elastomeric material may be composed of a variety of different insulating materials suitable for providing flexibility and insulation in accordance with this invention, such as latex rubber and the like. The electrode 74 may be a thin layer of electrically conductive but nonmetallic, flexible material may be disposed on all palm and finger surfaces of the glove 72.

Adjacent to the wrist portion of the glove 72, a connector 75 portion may be disposed of the same material as the electrode 74 and is integral with the electrode 74. The connector 75 portion may include one or more receiving apertures 76 adapted to accept lead wires 77a, 77b of a transcutaneous electrical nerve stimulation (TENS) unit 78 for the purpose of applying a combination of TENS and whole-hand massage (electro-massage) or acupuncture (electro-acupuncture) to a patient to reduce the pain to a patient's hand 30 or foot 31.

The electrode 74 may be fabricated from a thin layer of an elastomeric material such as carbonized rubber or carbonized silicone. The electrode 50 may be applied to the outer insulating portion 73 with an adhesive and/or it may be deposited as a coating.

TENS units are known in the art. According to this invention, the TENS unit 78 may be configured as a small, portable, battery operated device which emits biphasic pulses having zero net direct current. The amplitudes of the pulses may be up to sixty milliamps with durations of up to 500 microseconds and pulse rates of up to 200 hertz. The current emitted by TENS units which are powered by higher voltages, for example 220 volt AC, can be much greater than those previously described. For example, one type of AC powered TENS unit known as a high voltage pulsed galvanic stimulator (HVPGS), is capable of emitting pulses of up to 500 volts. Another type of TENS unit known as the Russian Faradic Unit, can emit pulses at rates up to 2000 hertz. A special class of TENS unit known as a Functional Electrical Stimulation (FES) unit, is a battery operated unit which is specifically designed to elicit timed cyclical contractions to strengthen muscles or prevent atrophy from disuse.

The electrode glove 72 according to the present invention provides for a method of combining the therapeutic effects of TENS with the benefit of manual massage when the glove is connected to a TENS, FES, or HVPGS unit.

In operation, one lead wire 77a from a TENS unit output channel is connected to energize the glove electrode 74, while the other lead wire 77b may be connected to energize a large indifferent electrode 79 attached to the patient. According to another aspect of this invention, as shown in FIG. 8, a second electrode glove 80 may be worn on the patient's other hand. The first glove 72 may be connected to the anode of a TENS unit output channel and the second glove 80 may be connected to the cathode of the channel. If more power than is normally available from a single channel of a TENS unit is needed, the lead wires 87 of similar polarity from each channel of a dual channel unit which has output channels that fire synchronously may be connected to a first glove electrode and the remaining lead wires of similar polarity may be connected to the second glove electrode or an indifferent electrode.

According to this configuration, a therapist or the patient can apply a deep electro-massage to substantially the entire hand. The stimulating electrodes can be significantly separated from each other to permit maximum current penetration and prevent shorting between the electrodes. Increases in current density are minimized which can result from small point type electrodes used in known devices.

Therapeutic Device Combined with Heat and Electrical Stimulation

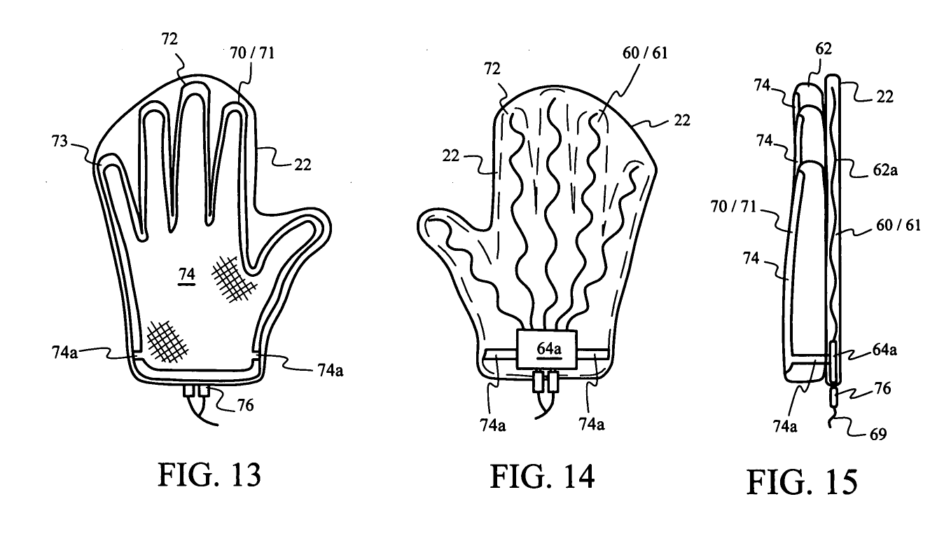

FIGS. 13, 14 and 15 show a therapeutic device 13 configured as a glove 62 operably integrating both an electrical mechanism 71 and a heating mechanism 61 into a single therapeutic stimulation system, such as the glove 72 illustrated. According to this embodiment, both heating and electrical stimulation is possible individually and/or simultaneously. Although shown as a glove 72, the therapeutic device 10 may be configured as a sock-like booty (similar to the embodiment shown is FIGS. 11 and 12) that operably integrates the electrical mechanism 71 and the heating mechanism 61 into a single therapeutic stimulation system so that both heating and electrical stimulation is also possible individually and/or simultaneously.

FIGS. 13-15 illustrate an electrode glove 72 including an electrode 74 surrounded by an outer insulating portion 73. The electrode 74 is connected to a plug portion 64a disposed adjacent to the heating mechanism 61 through electrode lead straps 74a that extend from the electrode 74 to the plug portion 64a. The plug portion 64a according to this embodiment is adapted to provide connectivity for both the electrical mechanism 71 and the heating mechanism 61 as a single therapeutic stimulation mechanism. The operative function for the electrical mechanism 71 and the heating mechanism 61 according to this embodiment function as described before but may be programmed to act separately, or in conjunction with each other, as desired by the user or person prescribing a particular mode of therapy.

As is known in the art, the electrical mechanism 71 and the heating mechanism 61 would be isolated from each other to prevent interference of their separate functions. Alternatively, the electrical mechanism 71 can be modified to provide heat (as well as to provide electrical pulse stimulation) as a heating mechanism to predetermined regions of the patient's hand 30 or foot 31.

According to this invention, a processor 67a may be implemented at various elements of this therapeutic device 10. The processor 67a may be implemented in association with, and/or as part of, the heating control portion 67, the electrical mechanism 71, the connectors 64, 75, and/or in association with any portion of the therapeutic device 10. The processor 67a may be implemented as a programmed general purpose computer, on a special purpose computer, a programmed microprocessor or micro-controller and peripheral integrated circuit elements, an ASIC or other integrated circuit, a digital signal processor, a hardwired electronic or logic circuit such as a discrete element circuit, a programmable logic device such as a PLD, PLA, FPGA or PAL, or the like. In general, any device processor capable of implementing the therapeutic functions shown in the figures can be used according to this invention. The particular form taken for the processor 67a is a design choice and will be obvious and predicable to those skilled in the art.

The processor 67a of the therapeutic device 10 can be associated with a storage unit (not shown) using any appropriate combination of alterable, volatile or non-volatile memory or non-alterable, or fixed, memory. The alterable memory, whether volatile or non-volatile, can be implemented using any one or more of static or dynamic RAM, a floppy disk and disk drive, a write-able or rewrite-able optical disk and disk drive, a hard drive, flash memory or the like. Similarly, the non-alterable or fixed memory can be implemented using any one or more of ROM, PROM, EPROM, EEPROM, an optical ROM disk, such as a CD-ROM or DVD-ROM disk, and disk drive or the like.

The processor 67a of the therapeutic device 10 is programmable and can be adapted to provide instruction to the therapeutic stimulation mechanism 60, 70. As mentioned previously, the therapeutic stimulation mechanism can include any one of, or both, the electrical stimulation mechanism 70 and/or the heating stimulation mechanism 60 in a single embodiment according to this invention. The processor 67a is programmable to provide predetermined amounts of heating and/or electrical simulation to the preferred body portion.

It will be recognized by those skilled in the art that changes or modifications may be made to the above described embodiment without departing from the broad inventive concepts of the invention. It is understood therefore that the invention is not limited to the particular embodiment which is described, but is intended to cover all modifications and changes within the scope and spirit of the invention.

What is claimed is:

1. A therapeutic device for biasing a body portion of a patient into a predetermined position, comprising:
a rigid support that is substantially flat and contoured to brace the body portion into the predetermined position;
a securing mechanism that braces the rigid support against the body portion; and
a therapeutic stimulation mechanism associated with the rigid support that is adapted to provide stimulation to the body portion.

2. The therapeutic device recited in claim 1, wherein the rigid support includes a cushion secured to the rigid support.

3. The therapeutic device recited in claim 1, wherein the rigid support includes a depression to align the body portion in a preferred orientation.

4. The therapeutic device recited in claim 1, wherein the body portion is at least one of: a hand and a foot.

5. The therapeutic device recited in claim 1, wherein the securing mechanism is a glove fastened to the surface of the rigid support.

6. The therapeutic device recited in claim 1, wherein the securing mechanism is a sock-like booty fastened to the surface of the rigid support.

7. The therapeutic device recited in claim 1, wherein the securing mechanism includes at least one of a strap, a buckle, a Velcro fastener, a clamp, and a tie.

8. The therapeutic device recited in claim 1, wherein the therapeutic stimulation mechanism is a heating device that provides heat stimulation to the body portion.

9. The therapeutic device recited in claim 1, wherein the therapeutic stimulation mechanism is integrated as part of the rigid support.

10. The therapeutic device recited in claim 1, wherein the therapeutic stimulation mechanism is integrated as part of the securing mechanism.

11. The therapeutic device recited in claim 8, wherein the heating device includes a plug connector portion into which a power supply is connected and from which wires extend to provide warmth along a length of the body portion by a heating control unit.

12. The therapeutic device recited in claim 8, wherein the heating device includes a programmable heating control unit that remotely controls warmth to the therapeutic device.

13. The therapeutic device recited in claim 8, wherein the heating device includes a programmable heating control unit that is controlled on the therapeutic device as a self-sustained therapeutic device.

14. The therapeutic device recited in claim 1, wherein the therapeutic stimulation mechanism is an electrical stimulation device to provide stimulation to the body portion.

15. The therapeutic device recited in claim 14, wherein the electrical stimulation device includes an outer insulating portion and a thin layer of electrically conductive material disposed on all palm and finger surfaces of a securing mechanism where the securing mechanism is a glove.

16. The therapeutic device recited in claim 14, wherein the electrical stimulation device includes an outer insulating portion and a thin layer of electrically conductive material disposed on an upper surface of the securing mechanism where the body portion is the foot and the securing mechanism is a sock-like booty.

17. The therapeutic device recited in claim 14, wherein the therapeutic stimulation mechanism includes a connector portion including a transcutaneous electrical nerve stimulation (TENS) unit that applies at least one of: a TENS, whole-hand massage unit (electro-massage), and an acupuncture unit (electro-acupuncture) to the patient to reduce the pain to the body portion.

18. A therapeutic device for biasing a body portion of a patient into a predetermined position, comprising:
a rigid support that is substantially flat and contoured to brace the body portion into the predetermined position;
a securing mechanism braces the rigid support against the body portion; and
a therapeutic stimulation mechanism includes an electrical stimulation mechanism and a heating mechanism to provide heating and electrical stimulation to the body portion.

19. The therapeutic device recited in claim 18, wherein the therapeutic stimulation mechanism includes a programmable processor adapted to provide predetermined amounts of heating and electrical simulation to the body portion.

20. The therapeutic device recited in claim 18, wherein the therapeutic device includes a plurality of therapeutic devices adapted for various body portions being operated simultaneously to provide predetermined amounts of heating and electrical simulation to the body portions.
